SEQUENCE LISTING (2 sequences)

SEQ ID NO: 1 - protein, 24 aa, Saccharomyces cerevisiae
EELLSKNYHLENEVARLKKGSGSG

SEQ ID NO: 2 - protein, 247 aa, synthetic construct
MGPDIVMTQSPSSLSASVGDRVTITCRSSTGAVTTSNYASWVQEKPGKLFKGLIGGTNNRAPGVPSRFSGSLIGDKATLTISSLQPEDFATYFCALWYSNHWVFGQGTKVELKRGGGGSGGGGSGGGGSSGGGSEVKLLESGGGLVQPGGSLKLSCAVSGFSLTDYGVNWVRQAPGRGLEWIGVIWGDGITDYNSALKDRFIISKDNGKNTVYLQMSKVRSDDTALYYCVTGLFDYWGQGTLVTVSS

(19) United States
(12) Patent Application Publication
(10) Pub. No.: US 2025/0333785 A1
(43) Pub. Date: Oct. 30, 2025

(54) METHODS OF TAGGING RNA MOLECULES ASSOCIATED WITH EPIGENETICALLY MODIFIED CHROMATIN IN LIVING CELLS

(71) Applicant: City University of Hong Kong, Hong Kong (HK)

(72) Inventors: Jian YAN, Hong Kong (HK); Ligang FAN, Hong Kong (HK)

(21) Appl. No.: 18/650,149

(22) Filed: Apr. 30, 2024

(51) Int. Cl. C12Q 1/6874; C12Q 1/28; C12Q 1/6804; C12Q 1/6806; C12Q 1/6813

(52) U.S. Cl. CPC C12Q 1/6874; C12Q 1/28; C12Q 1/6804; C12Q 1/6806; C12Q 1/6813

(57) ABSTRACT

A method of tagging RNA molecules involved in an epigenetically modified chromatin in real-time in living cells is provided. The method involves the introduction of an epigenetic reader module protein, an engineered ascorbate peroxidase, and a biotin-aniline probe into a living cell. Subsequently, the biotin-aniline probe is oxidized by the engineered ascorbate peroxidase, resulting in the formation of a covalent bond between the probe and an RNA molecule within the epigenetically modified chromatin, thereby generating a biotin-tagged RNA molecule. Finally, the biotin-tagged RNA molecule is purified for subsequent sequencing analysis.

### REFERENCE TO SEQUENCE DISCLOSURE

The sequence listing file under the file name “P3141US00_sequence listing.xml” submitted in ST.26 XML file format with a file size of 2.6KB created on Apr. 12, 2024 and filed on Apr. 30, 2024 is incorporated herein by reference.

### FIELD OF THE INVENTION

The present invention generally relates to the field of molecular biology. More specifically the present invention relates to identifying RNA molecules associated with epigenetically modified chromatin in living cells.

### BACKGROUND OF THE INVENTION

Transcriptional regulation, a key mechanism in determining cellular function and plasticity, relies on a complex interplay of genetic and epigenetic factors encoded within the genome. Among these factors, histone post-translational modifications (PTMs) exert profound influence on chromatin structure and gene expression. Histones, the core components of chromatin, act as scaffolds for DNA organization and undergo PTMs such as lysine methylation and acetylation, which are tightly linked to transcriptional activity. For instance, histone modifications like histone H3 lysine 4 monomethylation (H3K4mel) and H3 lysine 27 acetylation (H3K27ac) are often enriched at active gene enhancers, facilitating chromatin interactions and transcription factor binding. The precise deposition and removal of these histone modifications by enzymes such as histone lysine methyltransferases (KMTs) and histone lysine acetyltransferases (HATs) are critical for cellular homeostasis, development, and disease progression.

However, the mechanisms underlying the regulation of epigenetic modifications remain largely elusive. A burgeoning body of evidence suggests that noncoding RNAs (ncRNAs) play pivotal roles as epigenetic regulators. In addition to microRNAs (miRNAs), which directly target and silence genes involved in chromatin modification, long noncoding RNAs (IncRNAs) serve as crucial mediators in decoding chromatin epigenetic signatures. These IncRNAs interact with histone-modifying enzymes, often termed “histone writers,” “readers,” and “erasers,” thereby facilitating their recruitment to specific genomic loci for site-specific transcriptional regulation. Dysregulation of these IncRNAs is frequently associated with severe diseases. For instance, the IncRNA ST3Gal6-AS1 interacts with MLL1, promoting the deposition of the H3K4me3 mark at the ST3Gal6 promoter. During colorectal cancer progression, dysregulated expression of ST3Gal6-AS1 and ST3Gal6 activates the PI3K/Akt signaling pathway, driving tumor cell proliferation. Despite the recognized significance of IncRNAs as epigenetic regulators, only a small fraction of the vast array of IncRNAs encoded in the human genome have been identified and functionally characterized. This slow progress in elucidating epigenetic regulatory IncRNAs hinders their potential applications as diagnostic or prognostic biomarkers and therapeutic targets, underscoring the need for further research in this field.

Existing methods for interrogating noncoding RNAs associated with specific chromatin modifications include ChRIP-seq, CARIP-Seq, PIRCh-seq, and RT&Tag. While RT&Tag differs from the others, the former three methods share a similar principle, with minor variations primarily in the crosslinking process. CARIP employs 1% formaldehyde for crosslinking RNA-chromatin, with the reaction lasting only 1 minute, indicating a preference for milder crosslinking conditions. Consequently, the inventor reduced the number of sonication cycles, aiming to preserve more RNA-protein interactions. In contrast, ChRIP-seq incorporates additional ultraviolet (UV) crosslinking after a 10-minute treatment with 1% formaldehyde, coupled with intensive sonication, suggesting a belief in the efficacy of double crosslinking to retain RNA-chromatin interactions. However, the impact of crosslinking was not systematically discussed. Notably, the inventors of PIRCh-seq experimented with different crosslinkers and ultimately chose 1% glutaraldehyde as a replacement for formaldehyde, claiming improved capture of RNA-chromatin associations. Despite these efforts, the optimal crosslinking strategy remains inconclusive, underscoring the need for methods that bypass the crosslinking step altogether. Furthermore, a major limitation of these methods is their reliance on high-quality antibodies, typically validated as ChIP-grade. While histone PTM-specific antibodies are standard reagents, the variability in specificity and binding affinity across antibody lots can significantly impact data reliability. To ensure data integrity, the ENCODE consortium mandates multiple supporting characterizations, including dot blot assays, immunoblots, and mass spectrometry, for each antibody lot used in generating published data.

As a result, there is a pressing need for a method that can efficiently capture RNAs associated with various chromatin markers in living cells, circumventing the limitations posed by antibody dependence and crosslinking procedures. Therefore, the present invention addresses this need.

### SUMMARY OF THE INVENTION

It is an objective of the present invention to provide method, or kit to solve the aforementioned technical problems.

In accordance with a first aspect of the present invention, a method of tagging RNA molecules involved in an epigenetically modified chromatin in real-time in living cells is introduced. The method includes the following steps:

introducing an epigenetic reader module protein, an engineered ascorbate peroxidase and biotin-aniline probe into a living cell; oxidizing the biotin-aniline probe by the engineered ascorbate peroxidase and forming a covalent bond between the biotin-aniline probe and an RNA molecule associated with epigenetically modified chromatin to obtain a biotin-tagged RNA molecule; and purifying the biotin-tagged RNA molecule for further sequencing.

In accordance with one embodiment of the present invention, the epigenetic reader module protein locates a location of the epigenetically modified chromatin and drags the engineered ascorbate peroxidase to the location.

In accordance with another embodiment of the present invention, the

oxidation of the biotin-aniline probe occurs when the engineered ascorbate peroxidase is exposed to H₂O₂.

In accordance with one embodiment of the present invention, the epigenetic reader module protein is an evolutionarily conserved protein derived from natural proteins.

In accordance with one embodiment of the present invention, the epigenetic reader module protein comprises chromodomain from mammalian CBX7 or Drosophila Polycomb (dPC) for H3K27me3 (trimethyl-histone H3 lysine 27), chromodomain from mammalian CBX1 for H3K9me3 (trimethyl-histone H3 lysine 9), and PHD domain from mammalian TAF3 for H3K4me3.

In accordance with one embodiment of the present invention, the epigenetic reader module is further fused with a plurality of a repetitive peptide epitope and the engineered ascorbate peroxidase is fused with a single-chain variable fragment (scFv).

In some embodiments, the repetitive peptide epitope is GCN4 protein with the amino acid sequence of SEQ ID NO: 01; and the single-chain variable fragment has the amino acid sequence as listed in SEQ ID NO: 02.

In accordance with one embodiment of the present invention, the repetitive peptide epitope is recognizable by the scFV for recruiting the engineered ascorbate peroxidase.

In accordance with a second aspect of the present invention, a kit for tagging RNA molecules involved in an epigenetically modified chromatin in real-time in living cells is provided. Specifically, the kit includes an epigenetic reader module protein, an engineered ascorbate peroxidase, and a biotin-aniline probe.

In accordance with one embodiment of the present invention, the components of the kit are directly delivered into a living cell.

In accordance with another embodiment of the present invention, the engineered ascorbate peroxidase oxidizes the biotin-aniline probe when the engineered ascorbate peroxidase is exposed to H₂O₂.

In accordance with one embodiment of the present invention, the oxidized biotin-aniline probe forms a covalent bond to an RNA molecule of an epigenetically modified chromatin to generate a biotin-tagged RNA molecule.

In accordance with one embodiment of the present invention, the biotin-tagged RNA molecule is further purified and enriched.

In accordance with one embodiment of the present invention, the epigenetic reader module protein locates a location of the epigenetically modified chromatin and drags the engineered ascorbate peroxidase to the location.

In accordance with one embodiment of the present invention, the epigenetic reader module protein is an evolutionarily conserved protein derived from natural proteins.

In accordance with one embodiment of the present invention, the epigenetic reader module protein includes chromodomain from CBX7 or dPC for H3K27me3, chromodomain from CBX1 for H3K9me3, and PHD domain from TAF3 for H3K4me3.

In accordance with one embodiment of the present invention, the epigenetic reader modules are further fused with a plurality of a repetitive peptide epitope having the amino acid sequence of SEQ ID NO: 01 and the engineered ascorbate peroxidase is fused with a scFv having the amino acid sequence of SEQ ID NO: 02.

In accordance with one embodiment of the present invention, the repetitive peptide epitope is recognizable by the scFV for recruiting the engineered ascorbate peroxidase.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Embodiments of the invention are described in more details hereinafter with reference to the drawings, in which:

FIGS. 1A and 1B depict an outline of one embodiment of the method of the present invention, in which FIG. 1A schematically depict a diagram showing the approach and FIG. 1B is a scatter plots showing transcriptome profile (FPKM) of cells transfected with mCBX7 reader correlates with the profile of untreated HEK293T cells;

FIGS. 2A-2U depict the identification results of RNAs associated with H3K27me3 utilizing the method of the present invention with the reader protein of mCBX7 or dPC, in which FIG. 2A depicts a volcano plot of all RNAs associated with H3K27me3 modification using mCBX7; FIG. 2B is a volcano plot showing the enrichment of noncoding RNAs associated with H3K27me3 modification using mCBX7; FIG. 2C is a pie chart showing the number and percentage of coding and noncoding enriched genes using mCBX7; FIG. 2D depicts the boxplots showing the expression level (FPKM) of RNAs enriched or not enriched by mCBX7 (left) and dPC (right) reader; FIG. 2E (dPC) displays a volcano plot of enriched all RNAs associated with H3K27me3 modification utilizing dPC; FIG. 2F (dPC) displays a volcano plot of enriched ncRNAs associated with H3K27me3 modification utilizing dPC; FIG. 2G is a 3D pie chart showing the number and percentage of coding and noncoding genes identified with dPC reader protein; FIG. 2H is a donut chart showing the proportion of the read results aligned to feature regions classified as exonic, intronic and intergenic; FIG. 2I is a donut chart depicting the proportion of the read results using dPC reader, aligned to feature regions classified as exonic, intronic and intergenic; FIG. 2J depicts the western blot of H3K27me3 in input and streptavidin-enriched samples; FIG. 2K is a Venn diagram revealing the comparison of H3K27me3 associated ncRNAs found by mCBX7 and dPC; FIG. 2L and FIG. 2M respectively show the validation of candidate genes by RIP-qPCR with RNAs enriched by using H3K27me3 (FIG. 2L) and EZH2 (FIG. 2M) antibodies; FIG. 2N and FIG. 20 respectively depict the RIP-qPCR validation of candidate genes in HCT116 cells with enriched RNAs using H3K27me3 (FIG. 2N) and EZH2 (FIG. 20) antibodies; FIG. 2P depicts the validation of knocking down efficiency of IncRNAs SETD5-AS1 and LINC00641 in HEK293T cells; FIG. 2Q shows the profile plots (upper) and heatmaps (lower) representing ChIP-seq signals upon depletion of LINC00641 or SETD5-AS1 around H3K27me3 peaks; FIG. 2R depicts the western blot of H3K27me3 and H3 in total protein from the cell depleted of the SETD5-AS1 (ASETD5-AS1) and LINC00641 (ALINC00641) using antisense oligonucleotides (ASOs); FIG. 2S depicts the confocal fluorescent images showing the comparison of the distribution of H3K27me3 in nucleus of cells deficient of the IncRNAs SETD5-AS1 (ASETD5-AS1) and LINC00641 (ALINC00641) to ASO control (NC) with partially enlarged images on the upper right corner; FIG. 2T shows the genome browser shots displaying the distribution of ChIP-seq (upper) and RNA-seq (lower) signals from samples of negative control (NC), LINC00641 knockdown (ALINC00641), SETD5-AS1 knockdown (ASETD5-AS1); and FIG. 2U depicts the profile plot showing the ChIP signals of H3K27me3, H3K9me3 and H3K4me3 surrounding the 1-kb upstream and downstream regions around the gene bodies (between TSS and TES) with shaded areas indicating standard error;

FIGS. 3A-3Q depict the identification results of RNAs associated with H3K9me3 utilizing the method of the present invention with the reader protein of mCBX7, in which FIG. 3A shows that RNAs are enriched by the mCBX1 reader and the volcano plot exhibiting the enrichment of noncoding RNAs associated with H3K9me3 modification; FIG. 3B depict the western blot of H3K9me3 in input and streptavidin-enriched samples; FIG. 3C is a volcano plot of all RNAs associated with H3K9me3 modification; FIG. 3D is a 3D pie chart showing the number and percentage of coding and noncoding enriched genes; FIG. 3E is a donut chart showing the proportion of the read results aligned to feature regions classified as exonic, intronic and intergenic; FIG. 3F depicts the validation of candidate genes by RIP-qPCR with RNAs enriched by H3K9me3 antibody; FIG. 3G reveals a RIP-qPCR validation of candidate genes in HCT116 cells with RNAs enriched by H3K9me3 antibodies; FIG. 3H depicts the validation of candidate genes by RIP-qPCR with RNAs enriched by SUV39H1 antibody; FIG. 3I shows a RIP-qPCR validation of candidate genes in HCT116 cells with RNAs enriched by SUV39H1 antibodies; FIG. 3J provide profile plots (upper) and heatmaps (lower) representing ChIP-seq signals upon depletion of LNC00662 around H3K9me3 peaks; FIG. 3K depicts the validation of knockdown efficiency of LINC00662 in HEK293T cells; FIG. 3L depicts the western blot of H3K9me3 and H3 in total protein from the cell depleted of the LINC00662 (4662) using ASOs; FIG. 3M displays the confocal fluorescent images showing the comparison of the distribution of H3K9me3 in nucleus of cells deficient of the IncRNA LINC00662 (ALINC00662) to ASO control (NC) with partially enlarged images on the upper right corner; FIG. 3N shows the genome browser shots revealing the distribution of ChIP-seq (upper) and RNA-seq (lower) signals from samples of negative control (NC), LINC00662 knockdown (ALINC00662); FIG. 30 shows the profile plot displaying the ChIP signals of H3K9me3, H3K27me3 and H3K4me3 surrounding the 1-kb upstream and downstream regions around the gene bodies (between TSS and TES), where the shaded areas indicating standard error; FIG. 3P is a boxplot showing the expression level (FPKM) of RNAs enriched or not enriched by mCBX1 reader in HEK293T cells; FIG. 3Q is Venn diagram showing common regulators of H3K27me3 and H3K9me3; and

FIGS. 4A-4L depict the comparison results among different methods, in which FIG. 4A depicts the volcano plot showing all of the enriched RNAs associated with H3K4me3 modification using the present identification method with mTAF3 reader and FIG. 4B depicts the volcano plot only showing the enrichment of noncoding RNAs; FIG. 4C is a 3D pie chart showing the number and percentage of coding and noncoding genes enriched by the present identification method with mTAF3 reader; FIG. 4D is a Donut chart showing the proportion the read results of mTAF3 reader aligned to feature regions classified as exonic, intronic and intergenic; FIG. 4E depicts the profile plot showing the ChIP signal of H3K4me3 surrounding the 1-kb upstream and downstream regions around the gene bodies (between TSS and TES) with the shaded areas indicating standard error; FIG. 4F depicts a Venn diagram exhibiting common ncRNAs between the present identification method with mTAF3 reader and H3K4me3 antibody-based PIRCh-seq; FIG. 4G depicts the boxplot showing the expression of RNAs enriched by the present identification method with mTAF3 reader; FIG. 4H depicts the boxplot showing the expression of RNAs enriched by PIRCh-seq; FIG. 4I depicts the RIP-qPCR validation of genes enriched by the present identification method with mTAF3 reader in H9 cells, more particular the genes are found by both the present identification and PIRCh-seq; FIG. 4J depicts the RIP-qPCR validation of genes enriched by the present identification method with mTAF3 reader in HEK293T cells, more particular the genes are investigated by both the present identification and PIRCh-seq; FIG. 4K depicts the RIP-qPCR validation of genes enriched by the present identification method with mTAF3 reader in H9 cells; and FIG. 4L depicts the RIP-qPCR validation of genes enriched by the present identification method with mTAF3 reader in HEK293T cells.

### DETAILED DESCRIPTION

In the following description, methods, and/or kits of identifying RNA molecules associated with epigenetically modified chromatin in real-time in living cells and the likes are set forth as preferred examples. It will be apparent to those skilled in the art that modifications, including additions and/or substitutions may be made without departing from the scope and spirit of the invention. Specific details may be omitted so as not to obscure the invention; however, the disclosure is written to enable one skilled in the art to practice the teachings herein without undue experimentation.

The term “H3K4me3” used herein refer to an epigenetic modification to the DNA packaging protein Histone H3 that indicates tri-methylation at the 4th lysine residue of the histone H3 protein and is often involved in the regulation of gene expression. The name denotes the addition of three methyl groups (trimethylation) to the lysine 4 on the histone H3 protein. Likewise, the term “H3K9me3” is an epigenetic modification to the DNA packaging protein Histone H3. It is a mark that indicates the tri-methylation at the 9th lysine residue of the histone H3 protein and is often associated with heterochromatin. The term “H3K27me3” is an epigenetic modification to the DNA packaging protein Histone H3. It is a mark that indicates the tri-methylation of lysine 27 on histone H3 protein.

In accordance with a first aspect of the present invention, a method of identifying RNA molecules interacted with epigenetically modified chromatin in real-time in living cells is provided. Particularly, the method does not involve crosslinking and antibody usage.

The method provides a means to tag RNA molecules involved in epigenetically modified chromatin in real-time within living cells. Initially, an epigenetic reader module protein, alongside an engineered ascorbate peroxidase and biotin-aniline probe, are introduced into the living cell. Following cellular uptake, the engineered ascorbate peroxidase oxidizes the biotin-aniline probe upon exposure to hydrogen peroxide (H₂O₂), thereby facilitating the formation of a covalent bond between the probe and RNA molecules associated with epigenetically modified chromatin. This process yields biotin-tagged RNA molecules within the cellular environment. Subsequently, the biotin-tagged RNA molecules are purified from the cellular milieu to isolate them for further downstream sequencing analysis.

Furthermore, in some embodiments, the method incorporates an epigenetic reader module protein that acts to localize to specific regions of epigenetically modified chromatin within the cell, thereby guiding the engineered ascorbate peroxidase to these targeted locations. The epigenetic reader module protein may be derived from evolutionarily conserved proteins and can include chromodomains from CBX7 and Drosophila Polycomb (dPC) for recognizing H3K27me3, chromodomain from CBX1 for H3K9me3, and PHD domain from TAF3 for H3K4me3.

Moreover, the epigenetic reader module protein can be further modified by fusion with repetitive peptide epitopes, facilitating the recruitment of the engineered ascorbate peroxidase, which is itself fused with a single-chain variable fragment (scFv). This fusion of repetitive peptide epitopes with the epigenetic reader module protein ensures recognition by the scFV, thereby aiding in the targeted localization of the engineered ascorbate peroxidase to specific chromatin regions of interest within the living cell.

In some embodiments, the repetitive peptide epitope has the amino acid sequence: EELLSKNYHLENEVARLKKGSGSG (SEQ ID NO:01); and the scFV has the amino acid sequence:

(SEQ ID NO: 02)
MGPDIVMTQSPSSLSASVGDRVTITCRSSTGAVTTSNYASWVQEKPGKLF
KGLIGGTNNRAPGVPSRFSGSLIGDKATLTISSLQPEDFATYFCALWYSN
HWVFGQGTKVELKRGGGGSGGGGSGGGGSSGGGSEVKLLESGGGLVQPGG
SLKLSCAVSGFSLTDYGVNWVRQAPGRGLEWIGVIWGDGITDYNSALKDR
FIISKDNGKNTVYLQMSKVRSDDTALYYCVTGLFDYWGQGTLVTVSS

In accordance with a second aspect of the present invention, a kit for tagging RNA molecules involved in an epigenetically modified chromatin in real-time in living cells is provided.

The kit provides a comprehensive solution for tagging RNA molecules associated with epigenetically modified chromatin in real-time within living cells.

Comprising an epigenetic reader module protein, an engineered ascorbate peroxidase, and a biotin-aniline probe, this kit offers a versatile toolkit for researchers to investigate dynamic epigenetic processes. Upon delivery into living cells, the components of the kit act synergistically to facilitate the real-time tagging of RNA molecules. Specifically, the engineered ascorbate peroxidase is activated upon exposure to hydrogen peroxide (H₂O₂), which in turn oxidizes the biotin-aniline probe. This oxidative process leads to the formation of a covalent bond between the probe and RNA molecules associated with epigenetically modified chromatin, thereby generating biotin-tagged RNA molecules.

Furthermore, the epigenetic reader module protein plays a crucial role in guiding the engineered ascorbate peroxidase to specific locations within the cell where epigenetic modifications are occurring. Derived from evolutionarily conserved proteins, the epigenetic reader module protein includes chromodomains from CBX7 and Drosophila Polycomb (dPC) for recognizing H3K27me3, chromodomain from CBX1 for H3K9me3, and PHD domain from TAF3 for H3K4me3. Moreover, the epigenetic reader module protein may be further modified by fusion with repetitive peptide epitopes, allowing for enhanced recognition and recruitment of the engineered ascorbate peroxidase. This fusion strategy ensures precise localization of the enzymatic activity to specific chromatin regions of interest within the living cell environment.

Additionally, the kit enables purification and enrichment of the biotin-tagged RNA molecules, facilitating downstream sequencing and analysis. By providing a comprehensive toolkit for real-time tagging of RNA molecules associated with epigenetically modified chromatin, this kit empowers researchers to gain deeper insights into dynamic epigenetic processes and their functional implications within living cells.

In one embodiment, the method is based on proximity biotinylation of nucleic acids through jointly applying the epigenetic reader module proteins with an engineered ascorbate peroxidase, for instance, APEX2, to living cells. APEX2 is able to catalyze the one-electron oxidation of biotin-phenol, a membrane permeable small molecule, to generate short-lived (<1 ms) highly reactive radicals that covalently conjugate onto protein or nucleic acids. It has been revealed that biotin-aniline (Btn-An) is a type of substrate of APEX2 but with higher reactivity than biotin-phenol in labelling RNA molecules on the presence of H₂O₂. The epigenetic reader modules are all derived from natural proteins which are evolutionarily conserved, and have been well validated in previous studies, thus possessing high affinity and specificity.

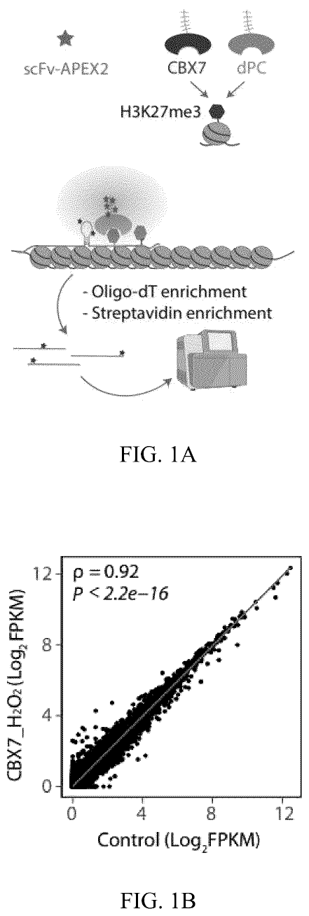

To enhance the labeling efficiency, a system called SunTag is further employed, in which the epigenetic reader module is fused with 10 copies of repetitive peptide epitopes having the amino acid sequence listed in SEQ ID NO: 01 recognized by the single chain Fv (scFv) with the amino acid sequence of SEQ ID NO: 02. As shown in FIG. 1A, instead of directly fusing APEX2 to the reader, scFv can bring up to ten APEX2 molecules to each piece of reader protein, markedly augmenting the focal concentration of the APEX2 enzyme (FIG. 1A). Given the high concentration and enzymatic activity of APEX2, the duration of efficient RNA labelling only took less than 1 minute. After Btn-An-labelling, the RNA is extracted and purified, and the biotinylated-RNA is subsequently enriched by streptavidin-coated beads. To further mitigate the noise from abundant ribosomal RNAs, polyA+RNA is enriched and primers with randomized sequences are used in the first strand synthesis of cDNA by reverse transcription, followed by template-switching and PCR amplification to generate the double stranded DNA library for high throughput sequencing. To call the significantly enriched RNAs, reader-free 10×SunTag is introduced with scFv-APEX2 to cells as control, carried out in parallel with the experiment. It is shown that the expression of these components and the introduction of Btn-An does not overtly affect the gene expression pattern in cells (FIG. 1B).

### EXAMPLES

### Example 1

### Identification of RNAs Associated with H3K27me3 Modification

The modification of H3K27me3, one of the most studied epigenetic modifications enriched in repressive regulatory elements. Two well-known distinct reader modules have been developed for ChromID, chromodomain in mammalian CBX7 protein (CBX7 hereafter) and drosophila Polycomb protein (dPC hereafter). First of all, both reader modules are revealed with high specificity to capture H3K27me3 modified histone in cells, with CBX7 showing higher affinity than dPC. Then, it is aimed to identify the RNA species that are associated with the modified chromatin.

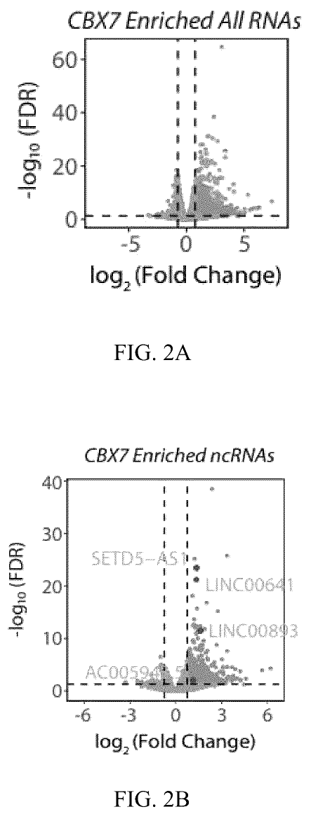

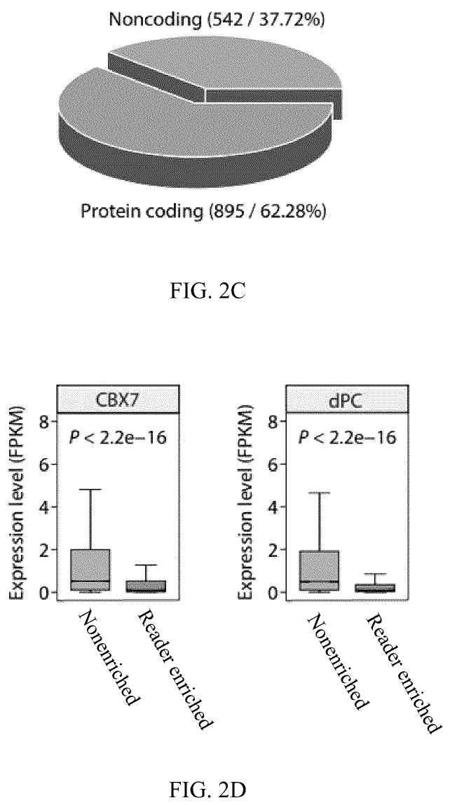

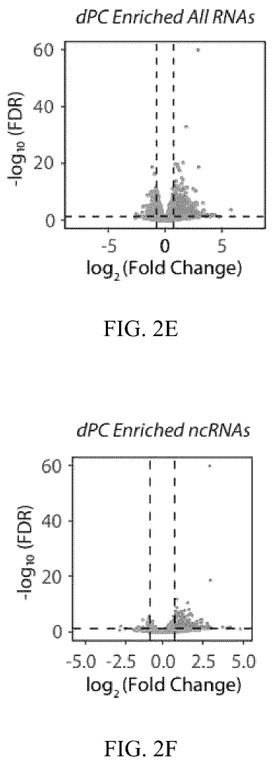

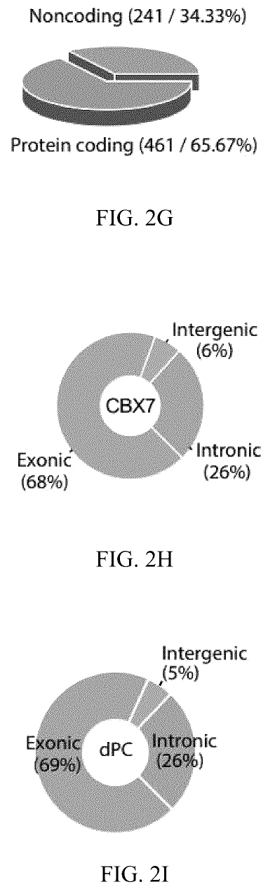

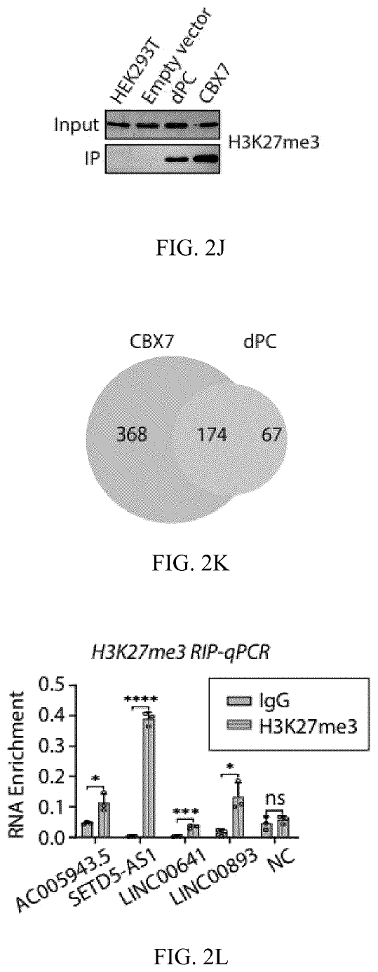

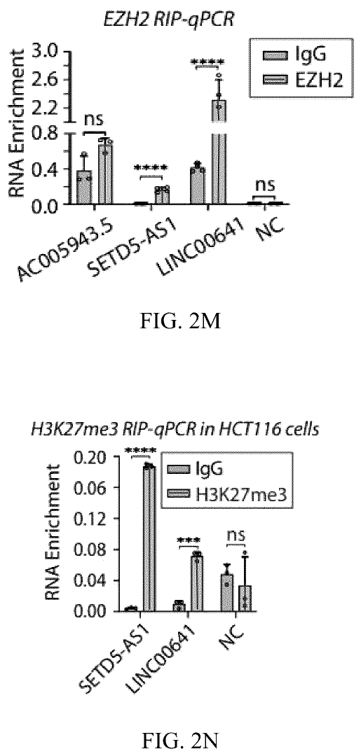

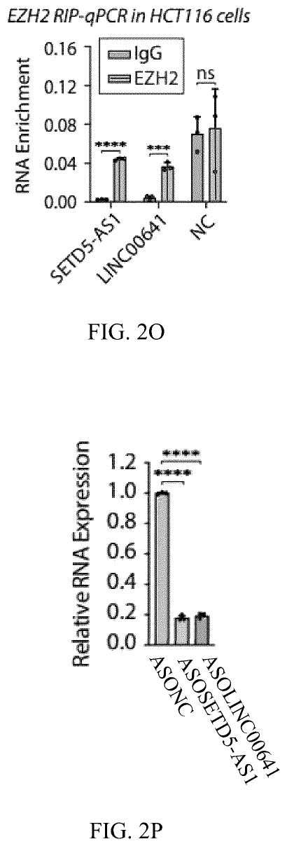

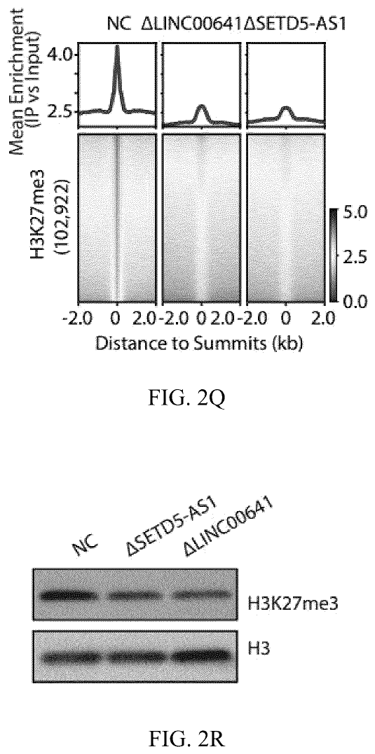

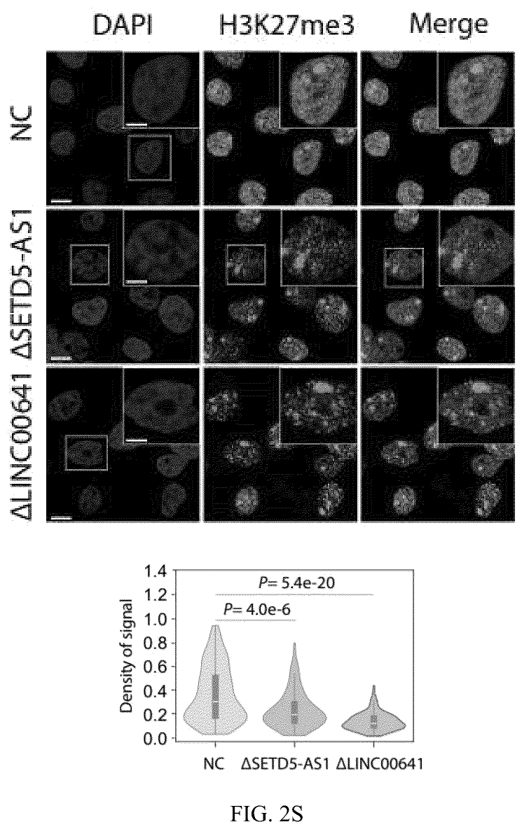

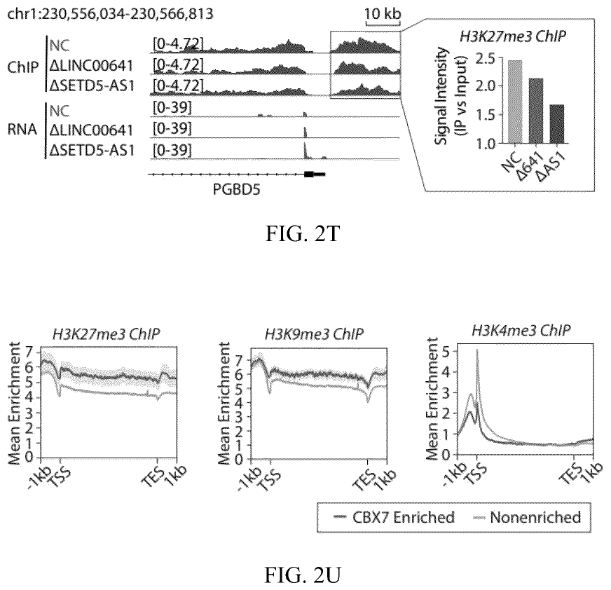

By applying the method of the present invention utilizing the CBX7 module as the epigenetic reader module protein, 1,437 different RNA species are identified to be significantly associated with H3K27me3 (log2-transformed fold change≥0.75, and False Discovery Rate, i.e., FDR<0.05; FIG. 2A), including 542 ncRNAs (taking up to ˜38% of all associated RNAs) (FIG. 2B and FIG. 2C). Among them, some of the well-established regulatory RNAs of H3K27me3 have been spotted, including those IncRNAs such as H1921, NEAT122, MALAT123, TINCR24 and SOX2-OT25. To exclude the possibility that the capture of these RNAs is caused by their high abundance in cells, it is shown that the RNAs enriched by the method display significantly lower expression level than the overall distribution of the cellular transcriptome (Mann-Whitney U test, P<2.2×10⁻¹⁶, FIG. 2D).

In comparison, utilizing dPC as the epigenetic reader module protein can only screen 702 RNAs significantly associated with H3K27me3 (FIG. 2E), of which 241 are noncoding (taking up to ˜34% of all associated RNAs) (FIG. 2F and FIG. 2G). In both experiments, the mapped reads are primarily aligned to exons (68% and 69% for CBX7 and dPC, respectively), with a small fraction of the intronic (26% and 26%) and intergenic (6% and 5%) origins (FIG. 2H and FIG. 21), demonstrating that the identified molecules are exclusively from RNA instead of genomic DNA contamination, an important indicator of the success of RNA capture. The smaller number of RNAs identified by dPC than CBX7 is highly likely due to its lower affinity to H3K27me3 revealed by the pulldown experiment (FIG. 2J). This is also supported by the fact that most (174/241) of the dPC-captured ncRNAs are already probed by CBX7 (FIG. 2K). Therefore, the subsequent analysis focuses on CBX7 data.

To characterize the functions of the ncRNAs in regulating epigenetic modification, a few moderately identified IncRNAs, encompassing AC005943.5, SETD5-AS1, LINC00641 and LINC00893 (FIG. 2B), are selected for molecular function validation. The interaction between these IncRNAs and H3K27me3 modified chromatin is firstly confirmed with RIP-qPCR. The abundantly expressed IncRNA BCYRN1 is used as negative controls. As expected, all of them display significant association except the negative control (FIG. 2L). In mammalian cells, the addition of H3K27me3 onto chromatin is mainly catalyzed by the conserved polycomb repressive complex 2 (PRC2), including the EZH1/2 catalytic subunit, SUZ12, EED, and RBBP7/4. Next, it is analyzed that if these IncRNAs are involved in facilitating the recruitment of enzyme responsible for deposition of H3K27me3. By performing RNA immunoprecipitation (RIP) using antibody specifically against the EZH2 catalytic subunit, two candidates, SETD5-AS1 and LINC00641, exhibit significant enrichment in the EZH2 pulldown RNAs (FIG. 2M), suggesting that they play a role in mediating the deposition of H3K27me3.

Furthermore, the role of these IncRNAs in other cell type is also assessed. RIP is conducted in HCT116 cells, a human colon cancer cell line, using the H3K27me3 and EZH2 antibodies. The qPCR results evidence that the association of SETD5-AS1 and LINC00641 to H3K27me3 (FIG. 2N) and EZH2 (FIG. 20), suggesting the conserved function of IncRNA across different cell types.

Next, ChIP-seq of H3K27me3 is performed in cells depleted of these IncRNAs using antisense oligos (ASO) (FIG. 2P). Surprisingly, almost all H3K27me3 peaks found in control cells are weakened when SETD5-AS1 or LINC00641 is attenuated (FIG. 2Q), revealing that the impact of these IncRNAs on H3K27me3 is conferred entirely for the whole genome. This is also consistent with the observations by Western blotting quantification (FIG. 2R) and immunofluorescent imaging (FIG. 2S). It is found that the knockdown of SETD5-AS1 or LINC00641 leads to a significant plunge of the signal of H3K27me3. To better illustrate whether the change influenced any transcriptional activity, a genome browser snapshot around the PGBD5 gene locus is taken as an example, showing that H3K27me3 signal around the gene PGBD5 promoter is notably reduced upon depletion of either IncRNA. Meanwhile, the downregulation of H3K27me3 is accompanied by reactivation of the PGBD5 gene (FIG. 2T). It has been reported that H3K27me3-associated RNAs are mostly transcribed from silenced genomic loci.

Indeed, we found that the RNAs enriched by the method of H3K27me3 are derived from repressive genomic regions decorated by higher H3K27me3 and H3K9me3 ChIP-seq signals but lower active H3K4me3 ChIP-seq signal at their transcription starting site (TSS) and gene bodies than nonenriched transcripts (FIG. 2U). These results demonstrated that the method of the present invention enables identification of IncRNAs associated with H3K27me3 signals. Some of these IncRNAs are indispensable for maintenance of the epigenetic modification and homeostasis of gene expression.

### Example 2

### Identification of RNAs Associated With H3K9me3 Modification

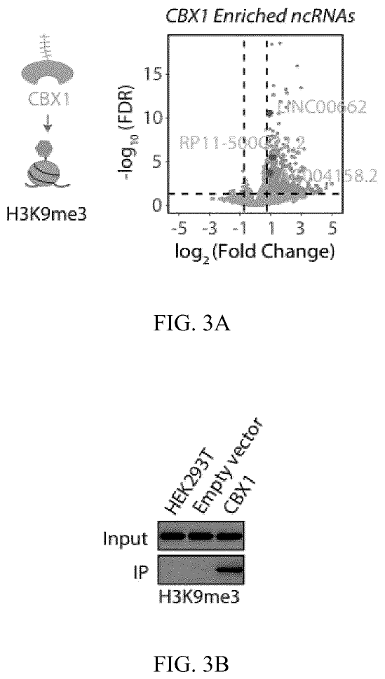

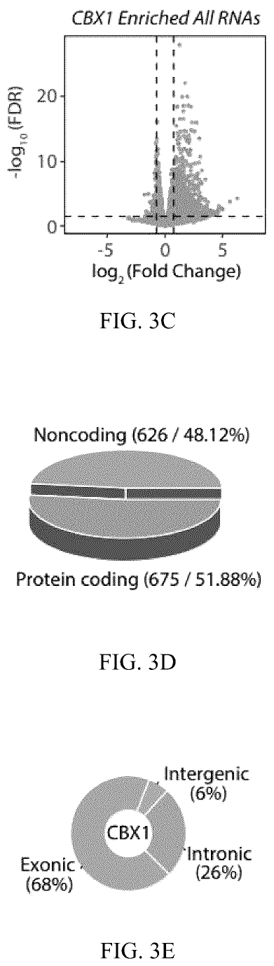

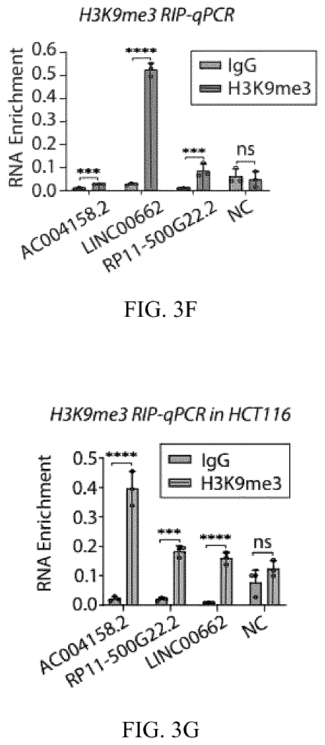

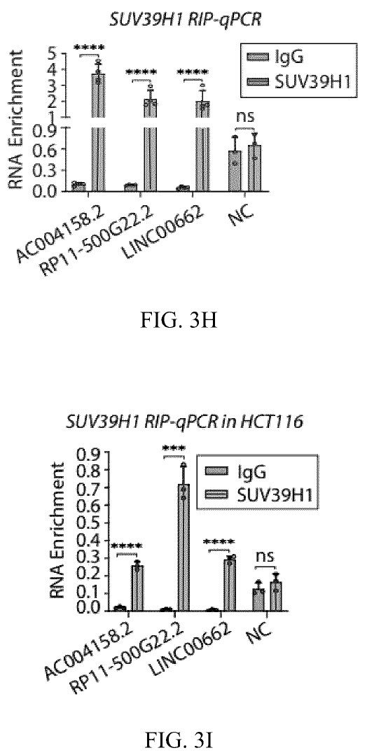

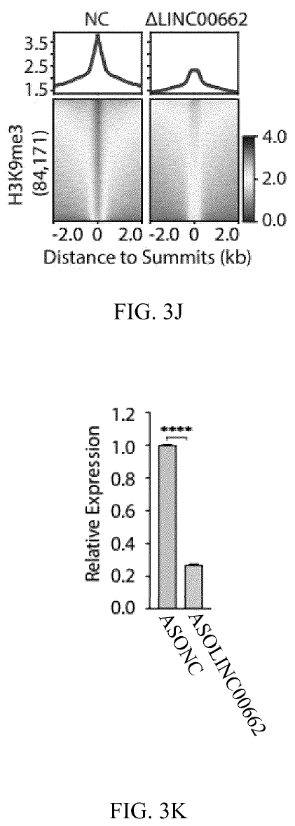

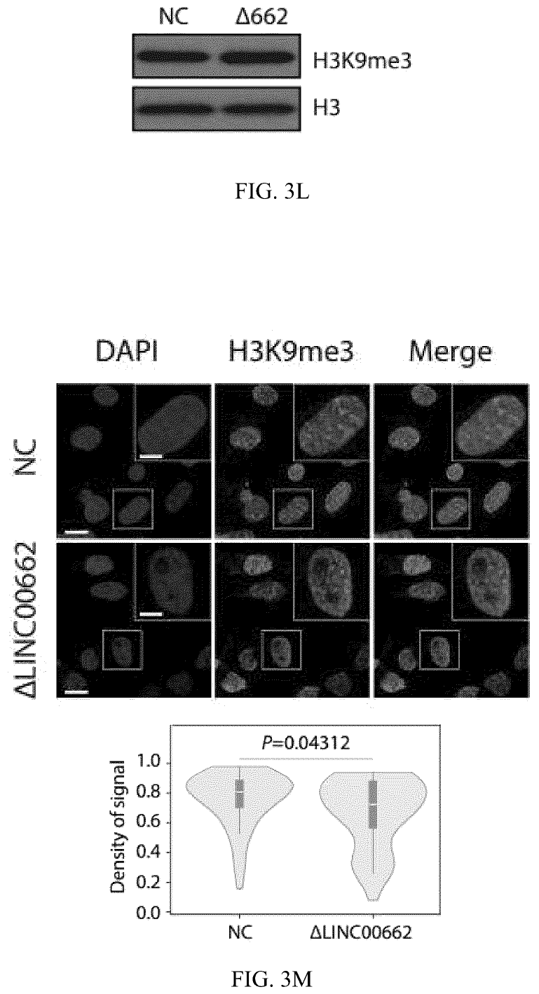

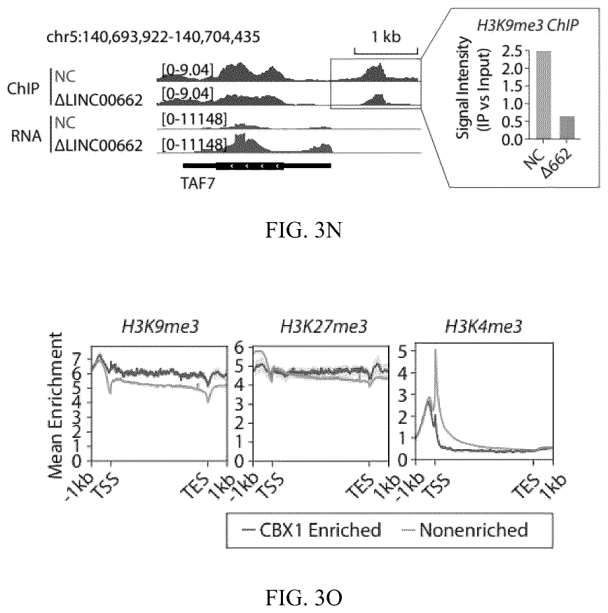

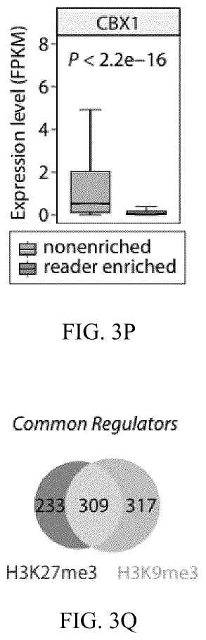

Furthermore, the method is applied to H3K9me3 using the reader module protein of CBX1 chromodomain (CBX1 hereafter) (FIG. 3A). First, the affinity of CBX1 to H3K9me3-decorated chromatin is confirmed by immunoprecipitation (FIG. 3B). The result of Chrom-seq showed that 1,301 RNA species are significantly associated with H3K9me3 (log2-transformed fold change ≥0.75, and FDR<0.05; FIG. 3C), including 626 ncRNAs (FIG. 3D). The protein coding genes took up approximately half of the enriched transcript species (FIG. 3D). Similar to H3K27m3 Chrom-seq, CBX1-captured reads were also mostly derived from annotated exons (68%), excluding the DNA contamination (FIG. 3E). Besides the known regulators of H19 and MALAT1, some novel IncRNAs are also identified, such as AC004158.2, LINC00662, and RP11-500G22.2 using RIP-qPCR in both HEK293T cells (FIG. 3F) and HCT116 cells (FIG. 3G). The deposition of H3K9me3 is catalyzed by a family of SET-domain containing methyltransferases, primarily SETDB1, SUV39H1 and SUV39H2. Thus, the interaction between SUV39H1 and the candidate IncRNAs is examined utilizing RIP-qPCR and the results show that all of them significantly binds to the SUV39H1 enzyme in both cell types (FIG. 3H and FIG. 31). One of the top enriched candidates, LINC00662, is selected for functional characterization. It is shown that H3K9me3 ChIP signal shows global reduction in the LINC00662 depleted cells compared to control (FIG. 3J and FIG. 3K). However, unlike H3K27me3, when cells are depleted of LINC00662, no overt change of H3K9me3 abundance is observed by Western blot analysis (FIG. 3L) or detected merely minor reduction by immunofluorescent imaging (FIG. 3M), suggesting that the impact of this IncRNA is more likely to be locus specific. For example, the enrichment of H3K9me3 at both the promoter and the gene body of TAF7 is remarkably dampened upon knockdown of LINC00662, consequently boosting the transcription of the nearby TAF7 gene (FIG. 3N). Similar to H3K27me3-associated RNAs, RNAs enriched around H3K9me3 are also transcribed from genomic regions with heavier modification of repressive marks, H3K9me3 and H3K27me3, and weaker H3K4me3 than the nonenriched transcripts (FIG. 30). This is also consistent with the fact that both H3K37me3 and H3K9me3associated RNAs show lower expression level than the overall transcriptome (FIG. 2D and FIG. 3P). Interestingly, when ncRNAs associated with both repressive marks are compared, more than one-third of them (309/859) exhibits linkage with both H3K9me3 and H3K27me3 (FIG. 3Q). Recently, evidences have emerged that these two repressive marks co-occurred at some developmentally repressed genes and transposable elements. And, the chromatin-associated proteomes of both marks also partially overlap, altogether suggesting the possible interplay between these epigenetic modifications in regulating gene silencing.

### Example 3

### Comparing The Performances of the RNA Identification Method of One Embodiment of the Present Invention to Other Methods

The major advancement of the present invention compared to the currently available methods to identify RNAs associated with epigenetic modifications, such as PIRCh-seq, ChRIP-seq, CARIP-seq or RT&Tag, is that the method and/or kits of the present invention no longer depends on antibody capture or covalent cross-linking, both of which are the major sources to cause experimental variation. Besides, the efficiency has also been significantly improved in terms of the number of captured IncRNAs, amount of input material, sequencing depth and cost (Table 1). RT&Tag, the most recently developed one, has remarkable advancement beyond other equivalent methods in many aspects, in particular its broad application, crosslinking-free nature and low cost. It claimed that as few as around 100,000 cells are needed for one assay which was more than 50-fold fewer than its counterparts. However, the demonstration of RT&Tag in the study has only been applied to the Drosophila cell that has a much smaller (>20 times) genome size (120 Mb) than the mammalian cells (1.6-6.3 Gb). More importantly, such a method still demands the high quality of antibody to recognize specific histone modifications, limiting its applicability.

TABLE 1
Comparison of the RNA identification method of one embodiment
of the present invention with other antibody-based methods

| Methods | The present method | | RT&Tag | | RIP-seq | | PIRch-seq | | ChRIP-seq | | CARIP-seq | |
|---|---|---|---|---|---|---|---|---|---|---|---|---|
| Input material | 0.5 | million | 0.1 | million | 10 | million | 4 | million | 20 | million | 10 | million |
| Cross-linking | No | | No | | Yes/No | | Yes | | Yes | | Yes | |
| Antibody | No | | Yes | | Yes | | Yes | | Yes | | Yes | |
| Sonication | No | | No | | Yes/No | | Yes | | Yes | | Yes | |
| Sequencing reads | 10-15 | million | 4-8 | million | 20 | million | 20 | million | 50 | million | 20 | million |
| Time | 2 | days | 1-2 | days | 3 | days | 3 | days | 3 | days | 3 | days |

| | | | | | | |
|---|---|---|---|---|---|---|
| Cost ($) | 25 | 50 | 150 | 225 | 200 | 200 |

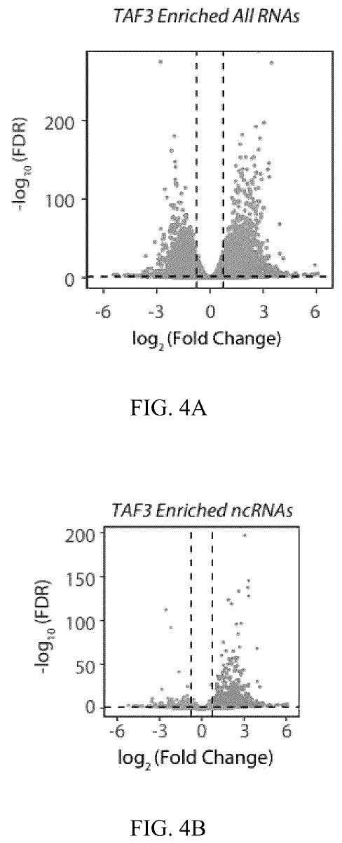

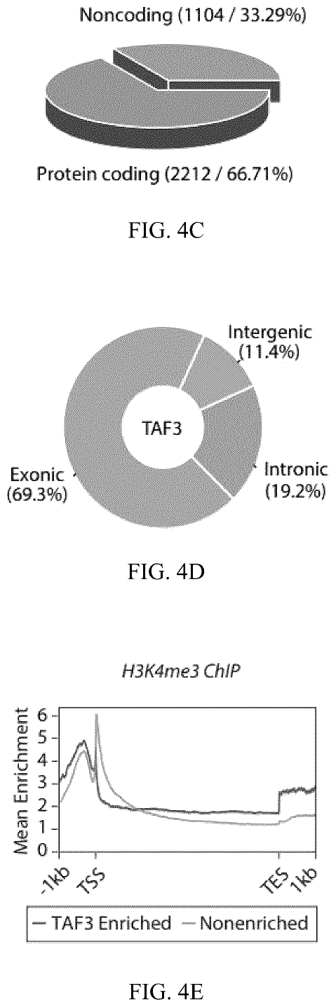

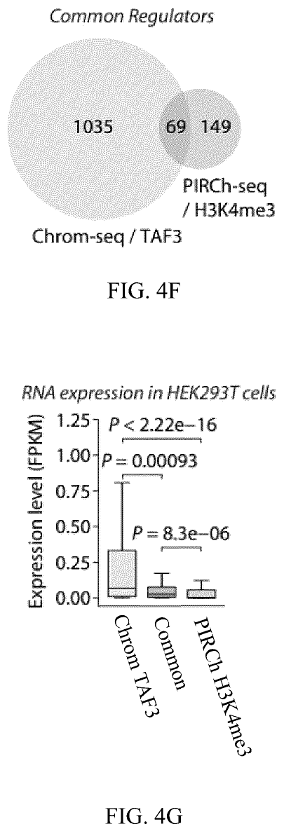

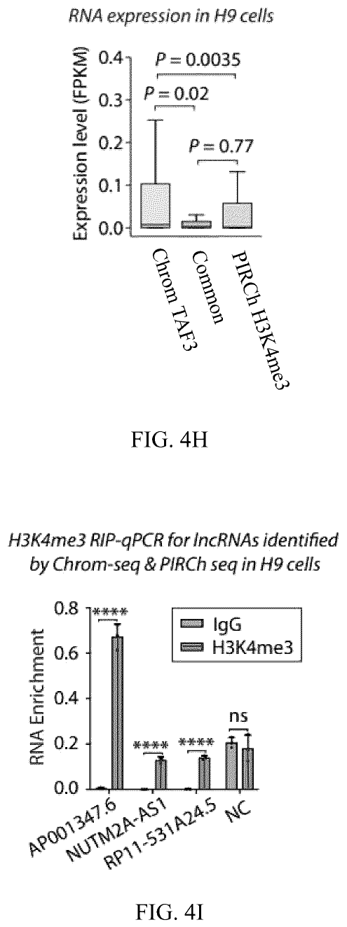

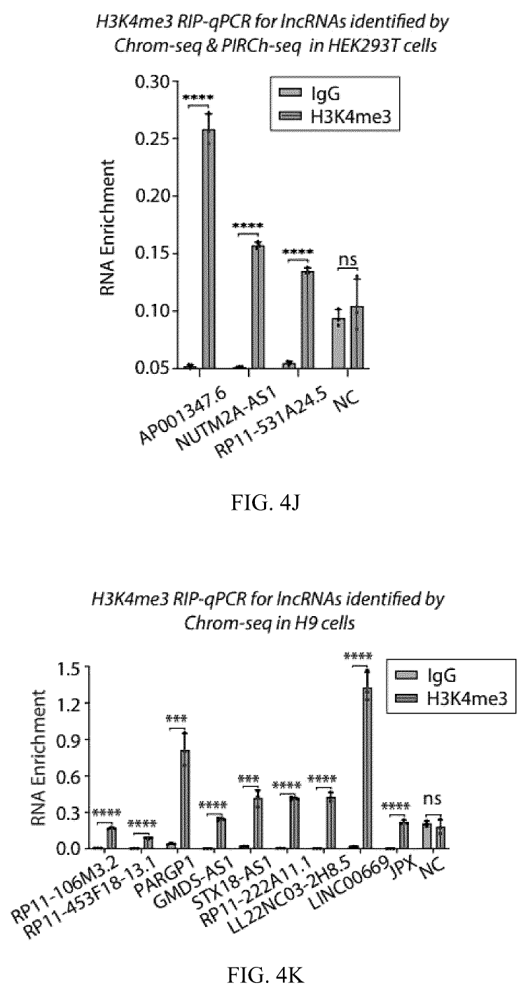

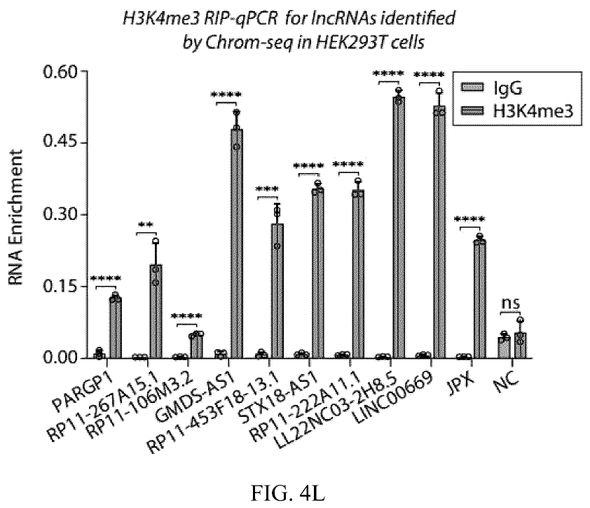

To demonstrate fair comparison of applicability in mammalian cells, the RNA identification method of the present invention is performed utilizing the PHD domain of mouse TAF3 (TAF3 hereafter) to target histone H3 lysine 4 trimethylation (H3K4me3), an epigenetic marker of the active promoter. As a result, the RNA identification method identifies 3,316 different RNA species (log2-transformed fold change >0.75, and FDR<0.05; FIG. 4A), including 1,104 ncRNAs (FIG. 4B and FIG. 4C). Likewise, the reads are mostly aligned to human exons (69.3%; FIG. 4D), verifying their derivation from RNA transcript. Given that H3K4me3 marks the active promoter which are close to TSS of the highly expressed genes, more RNAs are substantially identified than the other two repressive marks (3,316 vs 1,437 and 1,301). It is also observed that a significant enrichment of H3K4me3 signals is along with the gene body of the TAF3-enriched RNAs than the other genes (FIG. 4E), further suggesting that these RNAs are derived from H3K4me3-marked genes.

Meanwhile, the PIRCh-seq data of H3K4me3-enriched RNAs in a human embryonic stem cell line H913 is obtained. Based on the original publication, a very relaxed cutoff (log2-transformed fold change>0, and P≤0.05) is adopted to define the significantly enriched IncRNA. Even so, only 218 ncRNAs are detected, a number much lower than what Chrom-seq identified (1,104 IncRNA species) with a more stringent cutoff (log2-transformed fold change ≥0.75, and FDR<0.05). Although the two experiments are conducted in two different cells, the present method recovers one third of ncRNAs (69/218) found by PIRCh-seq in H9 cells (FIG. 4F). The failure of the present identification method in finding the remaining 149 H3K4me3-associated ncRNAs is most likely due to their extremely low expression level in HEK293T cells (median FPKM=0.0009; FIG. 4G). However, when the expression level of the three categories of genes in H9 cells is investigated, it is shown that some of the RNAs not-detected by PIRCh-seq are moderately or highly expressed (median FPKM=0.007; FIG. 4H), indicating that some IncRNAs missing by PIRCh-seq is possibly due to its low sensitivity. Therefore, some of the interactions in H9 cells are further examined with an orthogonal method, RIP-qPCR. First, it is confirmed that the H3K4me3-associated IncRNAs are found by both methods in HEK293T and H9 cells. As expected, all of them exhibits significant interactions in both cell types (FIG. 4I and FIG. 4J). Then, some positive examples with high expression in H9 cells that are from the present identification method but are not detected by PIRCh-seq are selected for validation with RIP-qPCR. The results show that these IncRNAs indeed interact with H3K4me3 in both HEK293T and H9 cells (FIG. 4K and FIG. 4L), recapitulating that the present invention outperforms other method in detecting IncRNA associated with chromatin epigenetic modifications, in addition to its independency of antibody or covalent crosslinking.

In summary, a novel method is introduced by the present invention to identify RNAs associated with various histone PTMs with the validation of the molecular function of some identified RNAs.

Outperforming the currently available methods, such as RIP-seq/CLIP-seq, PIRCh-321 seq/ChRIP-seq, and even the most recently developed RT&Tag, the method provided by the present invention demands remarkably lower amount of cells, sequencing reads, and thus strikingly lower cost. In addition, the method occurs under near-physiological conditions and avoids small molecule or UV-mediated crosslinking, dramatically reducing the nonspecific interactions. More importantly, ChIP-grade antibody is no longer required, replaced by the physiological reader domains of the targeted marks. On the one hand, this can avoid the large lot-to-lot quality variation of antibody that can lead to strong experimental noise, warned by ENCODE consortium and other scientists. On the other hand, the naturally occurring and engineered reader protein exceeds the antibody in terms of robustness, sensitivity and specificity, given the million years of evolution for precise control of epigenetic modifications and gene transcription. It is shown that Chrom-seq identifies >20 times the number of ncRNAs associated with the same H3K4me3 PTM than PIRCh-seq, caused by low efficiency of either crosslinking or antibody recognition. Other advantages include that: 1) protein engineering of the reading domains allow for the generation of higher affinity and specificity, recognition of novel modification such as m⁶A or Ψ, joint recognition of multiple different domains in one reaction, e.g. bivalent domains, and addition of fluorescent or affinity tags for simultaneous visualization; 2) lower cost, reduced from purchasing ChIP-grade antibody and sequencing, makes it feasible for scaling up the analyses in complicated biological systems. For instance, investigating the regulatory RNAs of various PTMs in a spectrum of tissues in an organism, or in cells under different circumstances.

As used herein, terms “approximately”, “basically”, “substantially”, and “about” are used for describing and explaining a small variation. When being used in combination with an event or circumstance, the term may refer to a case in which the event or circumstance occurs precisely, and a case in which the event or circumstance occurs approximately. As used herein with respect to a given value or range, the term “about” generally means in the range of ±10%, ±5%, ±1%, or ±0.5% of the given value or range. The range may be indicated herein as from one endpoint to another endpoint or between two endpoints. Unless otherwise specified, all the ranges disclosed in the present disclosure include endpoints. Furthermore, the terms “a” and “an” used herein are intended to be understood as meaning one or more unless explicitly stated otherwise. Moreover, the terms “first”, “second”, “third”, etc. are used merely as labels, and are not intended to impose numerical requirements on or to establish a certain ranking of importance of their objects.

The foregoing description of the present invention has been provided for the purposes of illustration and description. It is not intended to be exhaustive or to limit the invention to the precise forms disclosed. Many modifications and variations will be apparent to the practitioner skilled in the art.

The embodiments were chosen and described in order to best explain the principles of the invention and its practical application, thereby enabling others skilled in the art to understand the invention for various embodiments and with various modifications that are suited to the particular use contemplated.

1. A method of tagging RNA molecules involved in an epigenetically modified chromatin in real-time in living cells, comprising:
introducing an epigenetic reader module protein, an engineered ascorbate peroxidase and biotin-aniline probe into a living cell;
oxidizing the biotin-aniline probe by the engineered ascorbate peroxidase and forming a covalent bond between the biotin-aniline probe and an RNA molecule of an epigenetically modified chromatin to obtain a biotin-tagged RNA molecule; and
purifying the biotin-tagged RNA molecule for further sequencing.

2. The method of claim 1, wherein the epigenetic reader module protein locates a location of the epigenetically modified chromatin and drags the engineered ascorbate peroxidase to the location.

3. The method of claim 1, wherein the oxidation of the biotin-aniline probe occurs when the engineered ascorbate peroxidase is exposed to H₂O₂.

4. The method of claim 1, wherein the epigenetic reader module protein is an evolutionarily conserved protein derived from natural proteins.

5. The method of claim 4, wherein the epigenetic reader module protein comprises chromodomain from CBX7 or Drosophila Polycomb (dPC) for H3K27me3 (trimethyl-histone H3 lysine 27), chromodomain from CBX1 for H3K9me3 (trimethyl-histone H3 lysine 9), and PHD domain from TAF3 for H3K4me3.

6. The method of claim 4, wherein the epigenetic reader module is further fused with a plurality of a repetitive peptide epitope and the engineered ascorbate peroxidase is fused with a single-chain variable fragment (scFv).

7. The method of claim 6, wherein the repetitive peptide epitope is recognizable by the scFV for recruiting the engineered ascorbate peroxidase.

8. The method of claim 7, wherein the repetitive peptide epitope has an amino acid sequence of SEQ ID NO:01 and the scFV has an amino acid sequence of SEQ ID NO:02.

9. A kit for tagging RNA molecules involved in an epigenetically modified chromatin in real-time in living cells, comprising:
an epigenetic reader module protein;
an engineered ascorbate peroxidase; and
a biotin-aniline probe.

10. The kit of claim 9, wherein the components of the kit are directly delivered into a living cell.

11. The kit of claim 9, wherein the engineered ascorbate peroxidase oxidizes the biotin-aniline probe when the engineered ascorbate peroxidase is exposed to H₂O₂.

12. The kit of claim 11, wherein the oxidized biotin-aniline probe forms a covalent bond to an RNA molecule of an epigenetically modified chromatin to generate a biotin-tagged RNA molecule.

13. The kit of claim 12, wherein the biotin-tagged RNA molecule is further purified and enriched.

14. The kit of claim 9, wherein the epigenetic reader module protein locates a location of the epigenetically modified chromatin and drags the engineered ascorbate peroxidase to the location.

15. The kit of claim 9, wherein the epigenetic reader module protein is an evolutionarily conserved protein derived from natural proteins.

16. The kit of claim 9, wherein the epigenetic reader module protein comprises chromodomain from CBX7 or dPC for H3K27me3, chromodomain from CBX1 for H3K9me3, and PHD domain from TAF3 for H3K4me3.

17. The kit of claim 9, wherein the epigenetic reader modules is further fused with a plurality of a repetitive peptide epitope and the engineered ascorbate peroxidase is fused with a scFv.

18. The kit of claim 17, wherein the repetitive peptide epitope is recognizable by the scFV for recruiting the engineered ascorbate peroxidase.

19. The kit of claim 18, wherein the repetitive peptide epitope has an amino acid sequence of SEQ ID NO:01 and the scFV has an amino acid sequence of SEQ ID NO:02.
